(19) United States
(12) Patent Application Publication
(10) Pub. No.: US 2023/0149473 A1
(43) Pub. Date: May 18, 2023

(54) COMPOSITION COMPRISING CORD BLOOD PLATELET RICH PLASMA (CB-PRP) AND USES THEROF

(71) Applicants: Università Cattolica del Sacro Cuore, Milano (IT); Fondazione Policlinico Universitario Agostino Gemelli IRCCS, Rome (IT)

(72) Inventors: Alfonso SAVASTANO, Rome (IT); Nicoletta ORLANDO, Rome (IT); Caterina Giovanna VALENTINI, Rome (IT); Tomaso CAPOROSSI, Milano (IT); Stanislao RIZZO, Milano (IT)

(73) Assignees: Università Cattolica del Sacro Cuore, Milano (IT); Fondazione Policlinico Universitario Agostino Gemelli IRCCS, Rome (IT)

(21) Appl. No.: 18/056,624

(22) Filed: Nov. 17, 2022

Related U.S. Application Data
(60) Provisional application No. 63/264,185, filed on Nov. 17, 2021.

(51) Int. Cl. A61K 35/51; A61P 27/02; A61K 9/00; A61K 38/18

(52) U.S. Cl. CPC A61K 35/51; A61P 27/02; A61K 9/0048; A61K 38/18

(57) ABSTRACT

The present invention provides to compositions comprising cord blood platelet-rich plasma (CB-PRP) for the treatment of ocular diseases as well as methods of treatments of ocular diseases in which therapeutic effective amounts of blood platelet-rich plasma (CB-PRP) are administered to a subject in need thereof.

### TECHNICAL FIELD

The present invention relates to compositions comprising cord blood platelet-rich plasma (CB-PRP) for the treatment of ocular diseases as well as methods of treatments of ocular diseases in which therapeutic effective amounts of blood platelet-rich plasma (CB-PRP) are administered to a subject in need thereof.

### BACKGROUND

Ocular diseases or disorders are of a wide variety. Cataract, glaucoma, refractive errors are major causes of visual impairment and preventable blindness.

Notably, retinitis pigmentosa (RP) and atrophic age-related macular degeneration (d-AMD) represent a heterogeneous group of degenerations in which the genetic factor plays an important role. Together, both pathologies represent the major cause of blindness in the world. The genetic role is of considerable importance in both RP and d-AMD pathologies.

RP typically originates from genetic defects in rod photoreceptor proteins, resulting in night blindness in the early stages. Subsequently, the cone photoreceptors (responsible for day vision, visual acuity and colour vision) also slowly die, until all effective vision is lost. An alternative scenario implies the simultaneous involvement of both rods and cones, due to a genetic defect affecting both types of photoreceptors. Numerous molecular subtypes of RP are described, which are characterized by primary degeneration of the rods, followed by secondary dysfunction/degeneration of the cones; the loss of the cones in these cases occurs as a consequence of the loss of the rods and not as a direct consequence of a genetic mutation expressed in the cones (Narayan D S, et al, 2016). Therefore, a therapeutic intervention aimed at counteracting the secondary degeneration of the cones, is not influenced, or is only minimally influenced, in its effectiveness by the presence of a genetic defect selectively expressed in the rods.

D-AMD is a multifactorial disease in which genetic and environmental factors intervene. In the d-AMD, the deterioration of the retina is often associated with the formation of small yellowish deposits (lipofuscin), known as retinal drusen, below the retina in the macular region. This phenomenon leads to a thinning and drying out of the macula, causing the retinal macular tissue (atrophic development) to lose its function and central sight loss. The amount of central vision loss is directly related to the location of the retinal atrophic development. More and more genetic alterations are reported by the literature, although environmental factors have proved to play a key role in the phenotypic manifestation.

There is currently no cure for RP and d-AMD; however, several therapeutic strategies have reached the stage of clinical trials. One of these strategies is based on the use of neurotrophic factors, which act for their anti-apoptotic activity and their putative effects on the inflammation and energy metabolism of the cones. A large number of preclinical studies (Wen R, et al 2012; Earnings V, et al 2015) and clinical studies (Sieving P A et al 2006; Pilli S, et al, 2014; Falsini B et al, 2016) suggest a potential neuroprotective effect of the neurotrophins NGF, CNTF and BDNF on the survival of the retinal cones and on the tropism of the external retina in RP from primitive rod mutations. In particular, in the 6 months following treatment with neurotrophins, there is typically an increase in the thickness of the external retina, and specifically of the external nuclear layer (Wen et al, 2012, Sieving et al., 2006) which in a significant fraction of patients is associated with an improvement in visual acuity (Sieving et al, 2006), in the electroretinogram of the cones and in the Goldmann visual field. (Falsini et al 2016).

D-AMD also appears to have numerous clinical trials under evaluation. There are several studies about the opportunity to practice conservative therapies such as oral integration using antioxidants and anti-inflammatory treatment, but also interventional therapies such as sub-retinal transplantation of pigment epithelial cells.

Many are the efforts of scientists all over the world to try to combat the states of low vision/legal blindness related to diseases such as Retinitis Pigmentosa (RP) and even more so the Senile Macular Degeneration of the Atrophic Type (d-AMD). However, within this context, a dire need still exists to develop efficient therapeutic strategies for the treatment of such severe ocular diseases.

### SUMMARY OF THE INVENTION

The technical problem posed and solved by the present invention is hence that of providing an effective therapeutic approach for the treatment ocular diseases. The solution provided by the present invention is represented by a composition comprising cord blood platelet-rich plasma (CB-PRP).

As will be clearly evidenced in the experimental section of the present application, the authors of the invention have found that the administration of a composition comprising CB-PRP to a subject in need thereof, for example by way of sub-retinal injection, intravitreal injections or in the form of eye drops, depending on the disease to be treated, is particularly effective for the treatment of several ocular diseases, such as posterior segment eye diseases (PSEDs) as well as ocular surface diseases (OSDs).

Notably, sub-retinal administration of CB-PRP resulted in no serious adverse reactions such as endophthalmitis, retinal detachment, uveitis or haemorrhages, indicating the great potential of CB-PRP based compositions as safe products when placed in the contact with retinal tissue.

Hence, objects of the present invention are:
A composition comprising cord blood platelet-rich plasma (CB-PRP);
A composition comprising CB-PRP according to any of the embodiments disclosed in the present specification and in the claims for use in the treatment of an ocular disease;
A method for the treatment of an ocular disease in a subject in need thereof, which method comprises administering to said subject an effective amount of a composition according to any one of the embodiments disclosed in the present specification and in the claims;
The use of CB-PRP for the manufacture of a medicament, such as the composition according to any of the embodiments disclosed in the present specification and in the claims, for the treatment of an ocular disease.

Additional advantages and/or embodiments of the present invention will be evident from the following detailed description.

### BRIEF DESCRIPTION OF THE DRAWINGS

The present invention and the following detailed description of preferred embodiments thereof may be better understood with reference to the following figures:

FIG. 1: Prepared and thawed CB-PRP for intraocular use.

FIG. 2. CB-PRP aspirated into the sub-retinal injection syringe/pump.

FIG. 3. 41 G microneedle while performing sub-retinal injection of CB-PRP.

FIG. 4. OCT images showing patient #1's retina at baseline (upper figure) and 3 months (lower figure) post-retinal CB-PRP injection.

FIG. 5. Macular thickness analysis of the treated eye with high resolution optical coherence tomography (OCT). A: baseline. B: 3 months post-operative.

### GLOSSARY

As used herein, the term “cord blood platelet-rich plasma” (abbreviated as CB-PRP) is referred to plasma enriched in platelets obtained from umbilical cord blood. CB-PRP is a blood component for non-transfusion use.

As used herein, the expression “ocular surface disease” (OSD) comprises a spectrum of disorders that affect the normal structure and function of the cornea, conjunctiva, eyelids, and supportive glandular network. A significant proportion of such diseases have an immune aetiology, such as in allergic and autoimmune conditions.

As used herein, the expression “posterior segment eye disease” (PSED) comprises diseases of the retina, choroid and optic nerve of the eye.

In the present specification and claims, the expression “effective amount” is referred to a “therapeutically effective amount” of the compositions of the invention and is used to denote any amount which will cause a substantial improvement in the treated disease condition (such as a subsidence of a lesion, for example) when administered to the subject in need of treatment. As further explained below, the amount will vary e.g. depending on the condition being treated, the severity of the condition, the form and concentration of composition applied.

In any part of the present description and claims the term comprising can be substituted by the term “consisting of”.

### DETAILED DESCRIPTION

In the following, several embodiments of the invention will be described. It is intended that the features of the various embodiments can be combined, where compatible. In general, subsequent embodiments will normally be disclosed with respect to the differences with the previously described ones.

As previously mentioned, a first object of the present invention is represented by a composition comprising cord blood platelet-rich plasma (CB-PRP). According to the invention, said composition comprising CB-PRP is a hemoderivate composition.

According to the invention, CB-PRP may be obtained from umbilical cord blood by means of a method suitably selected by the skilled person among those that are already known in the art.

In one preferred embodiment of the invention, CB-PRP is prepared according to the procedure as defined by the Italian legislation on blood components as set forth in the following documents: “Decreto del Ministero della Salute, 2 Nov. 2015: Disposizioni relative ai requisiti di qualità GU n.300 del 28 Dec. 2015; Decreto del Ministero della Salute, 19 agosto 2019: Modifiche al decreto 2 Nov. 2015, recante: «Disposizioni relative ai requisiti di qualità e sicurezza del sangue e degli emocomponenti». GU n. 262 del Sep. 26, 2019” (herein incorporated by reference).

Merely by way of example, CB-PRP may be produced from umbilical cord blood by a method including the following steps:

i. preparing umbilical cord blood;

ii. isolating the platelet-rich plasma (PRP) fraction from the other blood components heavier than the PRP fraction.

Preferably, the isolation step ii. of the above method is performed by centrifugation, wherein the PRP fraction is isolated as the lighter component resulting from centrifugation.

According to one embodiment of the invention, CB-PRP is prepared from different cord blood units containing an adequate amount of hematopoietic progenitors. Preferably, CB-PRP is prepared from cord blood units collected from more donors according to any of the techniques known to a person skilled in the art, which units, after collection, are subjected to one or more safety screening tests so as to check the absence of fungi, aerobic and/or anaerobic bacteria in the same.

According to one preferred embodiment of the invention, CB-PRP is prepared from at least 15 units of umbilical cord blood collected from one or more donors, in particular by means of one or more centrifugation steps so as to isolate the PRP fraction from the other blood components. Preferably the CB-PRP is prepared from units of umbilical cord blood collected from different donors, preferably at least 15 different donors.

In one preferred embodiment, the collected blood cord units are subjected to a first “soft-spin” centrifugation step so as to obtain PRP, followed by a second “hard-spin” centrifugation step and by a third step of removal of the excess platelet-poor plasma.

It is preferred that all steps necessary to produce the CB-PRP occur in sealed bags, and that sterile connections are used to transfer the CB-PRP from a bag to another, or to store aliquots of the resulting CB-PRP. Additional microbial test can be performed on the final CB-PRP product so as to check the absence of e.g. fungi, aerobic and/or anaerobic bacteria in the same.

The resulting CB-PRP obtainable according to any of the embodiments described herein can be in liquid form, in solid form (also as a gel) or in freeze-dried form and can be stored in any device known in the art for storing platelet fractions or plasma.

The CB-PRP prepared according to any of the techniques known in the art or to any of the methods described herein can be aliquoted in sterile sealed tubes and stored at −80° C. before use.

According to one embodiment of the invention, the composition comprising CB-PRP disclosed herein is characterized by a platelet concentration of 1×10⁶ platelets/μL.

The composition of the invention comprises one or more platelet derived growth factors such as Granulocyte colony-stimulating factor (G-CSF), Granulocyte-Macrophage Colony-Stimulating Factor (GM-CSF), Vascular-Endothelial Growth Factor (VEGF), Fibroblast Growth Factors (FGF), Platelet Derived Growth Factor (PDGF).

One aspect of the invention is referred to a composition according to any of the embodiments described herein, further comprising at least one pharmaceutically acceptable excipient and/or carrier.

Preferably, a composition comprising CB-PRP according to any of the embodiments disclosed herein is a pharmaceutical composition.

Depending on the final form of the composition the skilled person will easily select suitable carrier/s and/or excipient/s. Suitable excipients and/or carriers that can be used for the preparation of a composition according to the present invention can be selected from those commonly used in the art such as, for example, stabilizers, preservatives, solvents, pH regulators, isotonic regulators, chelating agents, cryoprotective agents, diluting agents, binding agents, antioxidants, surfactants.

The compositions of the invention may further contain other active substances such as e.g. other hemostatic agents, antioxidants agents, neurotrophic factors, vitamins or molecules for the preparation of specific ocular formulations.

The compositions of the invention can be by way of example in the form of a solution, suspension, gel, eye drops.

When in the form of eye drops the compositions of the invention may particularly comprise preservatives and/or buffers so as to control the pH of the composition. Preferably, the pH of the composition of the invention in the form of eye drops equals that of tear fluid and is about 7.4.

As previously mentioned, one further aspect of the invention is related to a composition according to any of the embodiments described herein for use in the treatment of an ocular disease.

Advantageously, the compositions of the invention can be effectively used to treat an ocular disease as well as to prevent, reduce and/or eliminate any of the symptoms caused or associated to said ocular disease.

In some embodiments, said ocular disease is an ocular surface disease (OSD) or a posterior segment eye disease (PSED).

Non-limiting examples of OSD include Dry Eye Disease (DED), blepharitis and meibomian gland dysfunction (MDG), allergic eye diseases (AED), keratitis, microbic and/or neurotrophic corneal ulcers while examples of PSED include glaucoma, age-related macular degeneration (AMD), diabetic retinopathy (DR), uveitis and retinal vassal occlusions.

Preferably, said ocular disease is selected from the group comprising retinitis pigmentosa (RP), dry age-related macular degeneration (d-AMD), glaucoma disease and neurotrophic corneal ulcers.

The compositions according to any of the embodiments described herein can be administered by way of a topic and/or intraocular administration. According to some preferred embodiments, the compositions of the invention are administered by means of sub-retinal injection or intravitreal injection.

Such administration may be carried out by a surgeon or doctor by using any suitable method known in the art, e.g. in the field of ophthalmology.

In one preferred embodiment, the compositions of the present invention can be administered topically to the eye of a subject in need thereof in the form of eye-drops, e.g. according to its formulation.

The effective amount of CB-PRP in the composition of the invention for the treatment of an ocular disease may vary depending upon the exact age, as well as weight and sex of the subject being treated.

According to one preferred embodiment, the compositions of the invention can be administered in three different ways according to the type of ocular disease as indicated below:
Eye drops in the case of ocular surface diseases;
Intravitreal injections in the case of retinal/macular disorders;
Sub-retinal injection in the case of retinal/macular disorders.

Preferably, when administered by way of topical administration, such as in the form of eye drops, the compositions of the invention can be administered at a dose regimen of 1 drop of composition for 6 times daily for 7 days to taper down, according to the clinical ophthalmic condition.

Preferably, when administered by way of intravitreal administration, the compositions of the invention can be administered at a dose regimen of 1 injection of 0.1 mL of CB-PRP composition monthly for the first 6 months, wherein said injection is optionally repeated according to the clinical ophthalmic condition.

Preferably, when administered by way of sub-retinal injection, the compositions of the invention can be administered at a dose regimen of 1 injection only of 0.5 mL of CB-PRP composition (booster), which can optionally be followed by intravitreal administration according to any of the embodiments described herein, according to the clinical ophthalmic condition.

In accordance with certain embodiments, the compositions of the invention can be administered one or more times daily to a subject in need thereof. For example, the compositions according to any of the embodiments of the present invention can be administered once daily, two times daily, or about three times daily, or more. In certain embodiments, the regimen of the compositions of the invention can be administered in a subject in need thereof for a prolonged period.

All the compositions described herein may be prepared by employing standard preparation techniques known in the pharmaceutical field. The compositions can also be provided already aliquoted in single dosages or in single dosage fractions.

The compositions comprising CB-PRP according to the present invention may be employed in a method of treatment of an ocular disease in a subject in need thereof. In some embodiments said method of treatment comprises administering an effective amount of said composition to a subject in need thereof.

It is hence a further object of the present invention a method for the treatment of an ocular disease in a subject in need thereof, which method comprises:

administering to said subject an effective amount of a composition according to any of the embodiments disclosed in the present specification.

As previously mentioned, an effective amount of the composition of the invention that can be administered to said subject will vary depending on the age, weight, sex of said subject as well as depending on the severity of the ocular disease.

Preferably, the composition is administered in step i. at a dose regimen of 0.5 mL per eye.

As already mentioned above, step said administering can be carried out by way of a topic and/or intraocular administration. Preferably, the administration is carried out by means of sub-retinal injection or intravitreal injection or by means of topical administration on the ocular surface.

Merely by way of example, intravitreal injection of CB-PRP can be carried out using a 30 G syringe injecting 0.1 ml of CB-PRP directly into the vitreous chamber passing by pars plana (3.5 or 4.0 mm from the sclero-corneal limbus).

Merely by way of example, a sub-retinal injection of CB-PRP can be performed under the retina through a 41 G cannula. The injection site can be identified by the surgeon and under intraoperative conditions in the retinal region located inside the major vascular arches (posterior pole).

An ocular disease which can be treated according to any of the methods of treatment disclosed in the present specification can be an ocular disease as previously defined in the present specification or in the claims. Preferably, said ocular disease is selected from the group comprising retinitis pigmentosa (RP), dry age-related macular degeneration (d-AMD), glaucoma disease and neurotrophic corneal ulcers.

According to the present invention, “a subject in need thereof” can be a subject, preferably a human, who has been diagnosed as being affected by an ocular disease such as a disease defined in the present specification.

When sub-retinal administration is used, according to one aspect of the invention, said subject in need thereof may undergo cataract surgery and/or vitrectomy prior to administration of the composition of the invention according to any of the embodiments described herein.

In other terms, the method of sub-retinal treatment according to any of the embodiments disclosed in the present specification may further comprise one or more step prior to the administration of the composition of the invention, wherein said subject is submitted to cataract surgery and/or vitrectomy. Cataract surgery and vitrectomy procedures can be performed by means of a method suitably chosen by the skilled person among those that are already known in the art. Merely by way of example, High Speed Plana Pars Vitrectomy (10,000 cuts/minute) combined with lens phacoemulsification and simultaneously PC-IOL implantation, when required, can be performed prior to said administration. CB-PRP sub-retinal injection is carried out during the same operation. The subject undergoing said surgical operations will preferably receive peribulbar anaesthesia 30 minutes before surgery (10 ml of ropivacaine combined with hyaluronidase 300 IU). Also, the surgical field will be prepared as for standard ocular surgery with the periocular skin and the lower conjunctival fornix will be prepared with 5% povidone-iodine.

The sub-retinal method of treatment according to any of the embodiments disclosed in the present specification can further comprise at least one of the following:

subjecting said subject to vitrectomy;

subjecting said subject to peripheral retinal photocoagulation;

applying to said subject scleroconjunctival closures by means of bipolar diathermy or single suture,
after the sub-retinal administration of the composition of the invention, e.g. the sub-retinal administration of the CB-PRP injection of the invention.

All the additional procedures above can be performed by means of any of the standardized procedures known to a person skilled in the art.

In one aspect, the method according to any of the embodiments disclosed herein can further comprise after said sub-retinal administration:

subjecting said subject one or more times to at least one of the following diagnostic techniques: optical coherence tomography (OCT), visual field, electroretinography in order to assess the effectiveness of the treatment and/or monitoring the trend of the disease during a period of time.

diagnostic check may be performed immediately after said sub-retinal administration or at one or more time period after said administration, for example 1 day, 3 days, 1 week, 1 month, 3 months, 6 months and/or 12 months after step said sub-retinal administration. Subjecting the treated subject to one or more of the above-mentioned diagnostics may be useful, for example, for analysing the thickness of the outer nuclear layer (ONL) of the photoreceptors of the subject after treatment with the composition, thus for evaluating the success of the treatment and/or for monitoring the trend of the disease.

When the composition of the invention is administered topically, the term “administration” encompasses a treatment regimen in which said composition is administered in daily dosage/s for a given period of time that will depend on the pathology to be treated and on the response of the treated subject.

When the composition of the invention is administered intravitreally, the term “administration” encompasses a treatment regimen in which said formulation is administered using a 30 G syringe injecting 0.1 ml of CB-PRP directly into the vitreous chamber passing by pars plana (3.5 or 4.0 mm from the sclero-corneal limbus).

It forms part of the present invention also the use of CB-PRP for the manufacture of a medicament such as the composition according to any of the embodiments disclosed in the present specification or in the claims for the treatment of an ocular disease.

In one embodiment, said manufacture may comprise one or more steps of preparation of the CB-PRP, e.g. according to any of the methods disclosed in the present specification. Said manufacture may also involve mixing CB-PRP with one or more excipient and/or carrier such as those disclosed in the present specification thereby obtaining a composition according to any of the embodiments described herein.

Examples are reported below which have the purpose of better illustrating the methodologies disclosed in the present description, such examples are in no way to be considered as a limitation of the previous description and the subsequent claims.

### Examples

Primary Goal

The primary objective of the study was the quantitative evaluation of images derived from high-resolution optical coherence tomography (structural OCT). In particular, the differences of the images obtained studied with the image subtraction software at 1, 3, 6 and 12 months for the analysis of the thickness of the outer nuclear layer (ONL) of the photoreceptors. These differences were correlated between the same eye and the untreated adelph.

Secondary Objectives
ETDRS visual acuity assessment at 1, 3, 6 and 12 months
Microperimetry evaluation at 6 and 12 months
Evaluation of focal electroretinogram (fERG) at 6 and 12 months [22], [23]
Contrast sensitivity evaluation (MARS tables) 6 and 12 months
Goldmann perimetry evaluation at 6 and 12 months
Choroidal thickness
The thickness of the retinal ganglion cell layer (RGCL) [24], [25], [26].

### Study Design.

Safety and efficacy study, prospective, non-randomized cohort, with blood component. Duration of the study: 12 months.

### Materials and Methods

Due to the peculiarity of the study and the lack of previous similar studies on which to base hypotheses, the study was considered pilot for which we set a total sample size of 20 (N=10 patients for RP and N=10 patients for d-AMD).

A complete general clinical and ophthalmological examination (including detailed family history, anterior segment biomicroscopy, ETDRS corrected visual acuity, direct and indirect ophthalmoscopy, intraocular pressure measurement) was performed on each patient upon enrollment.

The study was conducted in accordance with Good Clinical Practice, the ethical principles deriving from the Declaration of Helsinki and the current legislation on clinical trials. The regulatory framework to which the study referred is that relating to blood components for non-transfusion use, to which all the procedures described will be strictly adherent.

This study was approved for testing by the local ethics committee (PROT ID 3417) and was covered by a specific insurance policy (LLOYD'S A1202049503-LB), pursuant to Ministerial Decree Jul. 14, 2009.

Sensitive patient data was collected in the data collection form or case report form (CRF), reporting only the patient's initials and an identification number consisting of a specific and unique 2-digit number.

All the documentation is available, for any checks and controls upon request to view the documentation directly to the PI of the study.

### Safety

Primary outcome: absence of major ocular adverse events (bacterial or fungal septic endophthalmitis, retinal detachment, vitreous proliferative-fibrotic reaction with retinal traction, secondary glaucoma, ftisis bulbi, iris rubeosis). Absence of major general adverse events (changes in blood count and systemic inflammatory parameters: ESR, CRP)

### Effectiveness

The primary objective of the study was to identify whether the new therapy could be effective and thus guarantee personalized medicine. To estimate the efficacy, the variation of a score derived from the anatomical parameters of the structural OCT during the follow-up visits was considered.

To achieve this goal, it was important to identify structural changes in precise retinal anatomical components that play a key role in visual recovery. Among these we distinguish: 1) Ellipsoid zone (Ellipsoid zone), Outer Nuclear layer (outer nuclear layer) and Ganglion complex layer (layer of the ganglion cell complex).

Inclusion Criteria
RP retinal dystrophy diagnosed by genetic testing and dry-AMD diagnosed with standard imaging methods used in daily clinical practice.
Relatively preserved central retinal function (Goldmann field of view V/4e≤30° only for patients with RP)
ETDRS corrected visual acuity≥light perception);
No concomitant ocular (e.g. glaucoma, amblyopia) or systemic pathology that could result in a BIAS for primary endpoint assessment

Exclusion Criteria
Age<18 years
Pregnancy
Previous inflammatory/infectious events involving the eyes
Concomitant eye diseases such as glaucoma or retinal detachment
Ocular trauma or diabetes and diseases potentially harmful to the visual system, even in the absence of impairment at the time of recruitment

### Adverse Events

The occurrence of a sudden worsening of the disease and/or bacterial infections and/or inflammatory reactions reasonably connected to the treatment with CB-PRP in three consecutive patients constituted a fundamental rule of stopping the trial.

Preparation of Cord Blood Enriched with Platelet Plasma.

CB-PRP is a blood component for non-transfusion use produced according to procedures defined by the Italian legislation on blood components (Decree of the Ministry of Health, 2 Nov. 2015: Provisions relating to the quality requirements GU No. 300 of 28 Dec. 2015; Decree of the Ministry of Health, 19 Aug. 2019: Amendments to the decree of 2 Nov. 2015, containing: “Provisions relating to the quality and safety requirements of blood and blood components”. OJ no. 262 of Sep. 26, 2019). The basic materials for the production of CB-PRP are cord blood units collected at the UNICATT cord blood bank of the Policlinico Gemelli IRCCS Foundation. These units are allogeneic solidarity donations for haematological patients who are candidates for hematopoietic stem cell transplantation: in this field, the use of cord blood represents a consolidated therapeutic practice for decades of experience. Cord blood units that can be used for transplantation must contain an adequate amount of hematopoietic progenitors. Units that did not meet the cell thresholds established for transplantation were not frozen and, with the consent of the donors, were intended for further clinical and research use. According to the aforementioned Italian legislation, couples who want to donate cord blood obtain eligibility after a thorough medical consultation. Furthermore, immediately after collection, infectious screening tests are performed on the maternal blood sample (serology and genome for HIV, HBV, HCV, and serological test for syphilis) or on the unit (blood cultures for fungi, aerobic and anaerobic bacteria). Only units with negative tests were used for subretinal injection.

The CB-PRP was made up of a pool of 15 units. Shortly after collection, each unit was subjected to a “soft-spin” centrifugation to obtain CB-PRP: the platelet concentration was then determined which will be normalized to 1×10 \hat{ }9/L, through a “hard-spin” and subsequent removal of excess platelet-poor plasma. CB-PRP was then recovered and stored at −80° C. pending microbial testing. Once 15 CB-PRPs with negative tests were obtained, in order to avoid any disparity in the concentration of growth factors between the different units, they were thawed and collected in a single bag, suitable for the preparation of blood components for non-transfusional use. The pool was then fractionated into 1 ml aliquots in sterile sealed vials, which were stored again at −80° C. until use. This process made the intervention homogeneous for all patients who participated in the study. All steps required to prepare the CB-PRP pool were performed in sterile cryogenic blood component bags, and sterile connections were used to transfer CB-PRP from one bag to another and to fractionate the CB-PRP aliquots.

### Surgical Technique of Subretinal Injection of CB-PRP

The surgical approach was a 23 Gauge (G) (10,000 cuts/minute) High Speed Plana Pars Vitrectomy combined with lens phacoemulsification and PC-IOL implantation simultaneously when required. Constellation Vision System (Alcon Laboratories, Inc., Fort Worth, Tex.) was the tool used for all procedures. All patients received peribulbar anesthesia 30 minutes before surgery (10 ml of ropivacaine combined with hyaluronidase 300 IU). The periocular skin and the lower conjunctival fornix were cleaned with repeated passages of 5% iodo-povidone. As previously described, prior to introducing 23 G trocar cannulae, the conjunctiva is partially displaced to misalign the conjunctiva from the sclera and an angled incision was made. Three valved cannulas were positioned at 2 and 11 o'clock for service sclerotomies and one in the lower temporal sector for infusion. They were positioned 3.5 mm from the limbus. Phacoemulsification was performed through 2 corneal incisions: a main (tunnel) incision of 2.2 mm and a service incision of 0.9 mm. During the vitrectomy, all eyes received the detachment and removal of the posterior hyaloid membrane if it had not already been separated from the underlying retinal layers. A “core vitrectomy” was then performed. Diluted triamcinolone acetonide will always be used to highlight the residual vitreous.

The procedures described up to now are standardized procedures for cataract removal and vitrectomy.[27], [28]

A sub-retinal injection of 0.5 ml of CB-PRP was performed under the retina through a 41 G cannula.

The injection site was identified by the surgeon and under intraoperative conditions, in the retinal region located inside or outside the major vascular arches.

A complete vitrectomy and eventual peripheral retinal photocoagulation, where necessary, for suspected rhematogenous areas was then performed. The filtered air was used as an internal buffer. After removal of the cannula, sclerotomies were checked and, if necessary, scleroconjunctival closures were applied using bipolar diathermy or a single suture (Vicryl 7-0; Ethicon Inc.). After surgery, patients were recommended to maintain a prone position for several hours for the 3 days following surgery.

### Analysis Plan Statistics

Measurements of retinal thicknesses, electrophysiological results of the electroretinogram (ERG), visual evoked potentials (PEV) and visual acuity were analyzed by multivariate analysis of variance for repeated measures (ANOVA). A test of normality was performed preliminarily to confirm that the distribution of the data approximates the Gaussian one. The changes recorded in the various times of the trial (1-12 months) were compared both with the baseline measures with the changes recorded in the untreated contralateral eyes. In the main analyzes, a P value<0.05 was considered statistically significant. Multiple comparisons were evaluated by Tukey's post-hoc test.

### Results

Of the 20 eyes hypothesized for the study, we have currently enrolled 13 eyes from 13 patients and treated 7 eyes (4 patients with RP and 3 with d-AMD) selected at our retinal disease and maculopathy clinics currently followed in our outpatient clinic (age range: 18-68 years).

TABLE 1
Baseline parameters of patients with RP

| | | | | | | Flicker ERG 8 Hz | | | | Flicker PEV 8 Hz | | | |
|---|---|---|---|---|---|---|---|---|---|---|---|---|---|
| | | | BCVA | | | OD | OD | OS | | OD | OD | OS | |
| Cog | Nom | Lat | OD | OS | CMT | amp | noise | amp | OS noise | amp | noise | amp | OS noise |
| G. | P. | Od | 3 + | 1 + | 135 | 0.11 | 0.13 | 0.48 | 0.24 | 0.73 | 0.18 | 1.10 | 0.04 |
| | | | 30 | 29 | | | | | | | | | |
| D. | OR | Os | 24 | 19 | 164 | 0.75 | 0.02 | 0.78 | 0.00 | 1.79 | 0.14 | 0.15 | 0.11 |
| S. | No. | Os | 0 + | 0 + | 140 | 0.13 | 0.10 | 0.23 | 0.05 | 0.31 | 0.28 | 0.29 | 0.08 |
| | | | 0 | 0 | | | | | | | | | |
| G. | F. | os | 0 + | 0 + | 222 | 0.21 | 0.48 | 0.44 | 0.32 | 0.48 | 0.11 | 2.41 | 0.41 |
| | | | 0 | 12 | | | | | | | | | |

TABLE 2
1-month parameters of RP patients

BCVA

| Cog | Nom | Lat | OD | OS | CMT |
|---|---|---|---|---|---|
| G. | P. | Od | 0 + 0 | 2 + 30 | 148 |
| D. | OR | Os | 25 | 33 | 163 |
| S. | No. | Os | 0 + 0 | 0 + 0 | 135 |
| G. | F. | os | 0 + 0 | 0 + 8 | 205 |

TABLE 3
3-month parameters of patients with RP

| | | | | | | Flicker ERG 8 Hz | | | | Flicker PEV 8 Hz | | | |
|---|---|---|---|---|---|---|---|---|---|---|---|---|---|
| | | | BCVA | | | OD | OD | OS | | OD | OD | OS | |
| Cog | Nom | Lat | OD | OS | CMT | amp | noise | amp | OS noise | amp | noise | amp | OS noise |
| G. | P. | Od | 0 + | 2 + | 145 | 0.4 | 0.11 | 0.18 | 0.9 | 0.73 | 0.18 | 1.10 | 0.04 |
| | | | 0 | 29 | | | | | | | | | |
| D. | OR | Os | 23 | 25 | 182 | 0.75 | 0.02 | 0.78 | 0.00 | 1.79 | 0.14 | 0.15 | 0.11 |

TABLE 4
Baseline parameters of d-AMD patients

| | | | | | | Flicker ERG 8 Hz | | | | Flicker PEV 8 Hz | | | |
|---|---|---|---|---|---|---|---|---|---|---|---|---|---|
| | | | BCVA | | | OD | OD | OS | | OD | OD | OS | |
| Cog | Nom | Lat | OD | OS | CMT | amp | noise | amp | OS noise | amp | noise | amp | OS noise |
| T. | M. | Od | 0 + | 0 + | 100 | 3.49 | 0.19 | 4.57 | 0.04 | 1.19 | 0.22 | 1.41 | 0.22 |
| | | | 5 | 30 | | | | | | | | | |
| B. | M. | Od | 6 + | 25 | 165 | 2.11 | 0.11 | 3.52 | 0.78 | 6.15 | X | 3.84 | 0.16 |
| | | | 29 | | | | | | | | | | |
| R. | G. | Od | 20 | 30 | 230 | 3.63 | 0.45 | 0.93 | 0.23 | 1.81 | 0.58 | 5.42 | 0.38 |

TABLE 5
1-month parameters of d-AMD patients

BCVA

| Cog | Nom | Lat | OD | OS | CMT |
|---|---|---|---|---|---|
| G. | P. | Od | 0 + 17 | 3 + 26 | 80 |
| D. | OR | Od | 6 + 35 | 26 | 175 |
| S. | No. | Od | 21 | 29 | 190 |

Table Legend:
Cog=initial of the patient's surname
Nom=initial of the patient's Name
Lat=laterality of the treated eye
BCVA=Best Corrected Visual Acuity measured by reading the letters ETDRS (Early Treatment Diabetic Retinopathy Study)
CMT=Central Macular Thickness measured in microns with optical coherence tomography (OCT)—Solix Optovue.
ERG=Electroretinogram
PEV=Visual evoked potentials

No serious adverse reactions were found in all eyes of treated patients and who received subretinal CB-PRP. Specifically, uveitis, retinal detachment, vitreous haemorrhage and endophthalmitis were not observed in any eye. In all eyes we observed a regular postoperative course. As for the eyes of patients treated by RP, the results of visual acuity show an improvement at 3 months of patient n. 2 (DO) moving from a baseline of 19 letters read ETDRS to 33 letters ETDRS at 1 month and then 25 letters at 3 months. Patient no. 1 (GP), on the other hand, showed a decrease in visual acuity going from 3+30 letters ETDRS at baseline to 0+0 letters at 1 month and remaining at 0+0 letters ETDRS at 3 months. However, the patient felt satisfied as she reported seeing the colors of the brighter objects and better defining the contour of the objects at a distance (unfortunately these parameters are not well quantifiable with specific ophthalmic tests). The other 2 patients treated while maintaining a stable visual acuity at 1 month compared to the baseline (0+0 and 0+8 patient n.3 and n.4 respectively), were satisfied as they claimed to see better the colors of the objects. Regarding the analysis of the electrophysiological results, we did not record significant differences in ERG and VEP for the 2 patients who have a 3-month follow-up. Central macular thickness analysis showed no significant changes at either 1 or 3 months from baseline.

For the 3 eyes of patients suffering from d-AMD at 1 month after the injection we did not observe a significant increase in the number of letters read by the patients but also in this case the patients were not considered dissatisfied as they reported having, in some way, improved the quality of vision. Also in this case the analysis of the electrophysiological results of ERG, PEV and of the central macular thickness did not show significant changes at 1 month compared to the baseline.

### Discussion

In the 7 eyes we treated with subretinal CB-PRP, there were no serious adverse reactions such as endophthalmitis, retinal detachment, uveitis or haemorrhages. Regarding the parameters evaluated such as the electroretinogram and the visual evoked potentials, even if with a short follow-up, we did not observe significant changes compared to the baseline data, making us understand that CB-PRP is a safe product when placed on contact with the retinal tissue. The central retinal thickness (CMT) measured with modern high definition optical coherence tomographs (OCT), although showing a significant increase at 1 week, then approaches the baseline values already at 1 month in all patients. A careful analysis of the OCT images, both at 1 and 3 months (only 2 patients), shows a clearer stratification of the retina, especially of the outer layers. This finding could be compatible with the hypothesis that the growth factors contained in the CB-PRP could stimulate the retinal cells to realign themselves correctly in a retinal stratification that could be functional again. Instead, we reported conflicting results for what concerns visual acuity, that is, although all patients we injected report seeing better, we were not able to quantify this improvement. In one case (GP) we had a significant worsening of visual acuity after the injection but also in this case the patient reported seeing better the outlines of things and colors. Recently we are evaluating the importance of the different CB-PRP injection sites as we believe that the saving of the macular region from the retinal detachment bubble (which is obtained when the CB-PRP is injected below the retina) can bring the same number of neurotrophic factors, avoiding the trauma of the separation of the neuroepithelium of the retinal pigment epithelium in the macular region. Since CB-PRP had never been evaluated until now for what concerns the injection inside the eye and in the subretinal spaces, this treatment could probably find the maximum of its rationale for use in the early stages of the disease. The cases we treated, for precautionary reasons, were patients with very advanced pathology in both the RP and d-AMD groups. In the near future, the surgical approach could be different, preferring, for example, the intravitreal rather than the subretinal injection route given the great surgical experience and high-cost instrumentation required linked to the latter. It could also be important to anticipate the timing of the CB-PRP injection precisely in order to prevent the development of a disabling disease.

In conclusion, the subretinal injection of CB-PRP has been shown to be safe and without serious adverse reactions in the cases followed so far. Its real efficacy remains to be clarified by performing this injection in a greater number of patients and with longer follow-ups with regard to RP and d-AMD. The intravitreal route of administration remains an area still to be explored and which would entail greater practicality and ease of use of the CB-PRP for intraocular use.

It forms part of the present description also the following:

Administrative information

| | | |
|---|---|---|
| Title | 1 | Subretinal injection of cord blood-derived, growth factor- |
| | | enriched plasma, as a treatment strategy for non-cell |
| | | autonomous cone photoreceptor degeneration in retinitis |
| | | pigmentosa and dry age-related macular degeneration. The |
| | | SICord STUDY |
| Trial registration | 2a | Trial will be registered in ClinicalTRials.gov |
| | 2b | |
| Protocol version | 3 | Version 1, Mar. 1, 2020 |
| Funding | 4 | None |
| Roles and | 5a | Names, affiliations, and roles of protocol contributors |
| responsibilities | 5b | Name and contact information for the trial sponsor |
| | | NA |
| | 5c | |
| | 5d | This is a single center study. The steering committee is as follows; Prof. |
| | | Stanislao Rizzo, Dott Alfonso Savastano, Prof. Benedetto Falsini, Prof. |
| | | Enrica Strettoi. Prof Ornella Parolini. |
| | | Endpoint adjudication committee |
| Introduction | | |
| Background and | 6a | Retinitis pigmentosa (RP) represents a heterogeneous group of inherited |
| rationale | | disorders and one of the major causes of blindness in the world. RP |
| | | originates typically from defects in photoreceptor proteins: first rods are |
| | | affected and night blindness develops. Then, cones also die, until all |
| | | effective sight is lost. There is currently no cure for RP; yet, several |
| | | therapeutic strategies have reached the stage of clinical trials. One of these |
| | | strategies is based on the use of neurotrophic factors, acting for their anti- |
| | | apoptotic activity and their putative effects on inflammation and |
| | | photoreceptor energy metabolism (see Falsini, Sieving and Bush, 2016, for |
| | | a review).^{1, 2} In the RP genetic subtypes characterized by primary rod |
| | | degeneration, followed by secondary cone dysfunction/degeneration, the |
| | | cone loss occurs as a consequence of rod photoreceptor loss, and not as |
| | | a direct consequence of the rod gene mutation. Typical example is the |
| | | mutation in the Rhodopsin gene, causing a RP whose natural history is well |
| | | characterized (Cidecyian et al, 2003),³ with cone loss starting after >75% |
| | | of rods are lost. A therapeutic intervention aimed at rescuing cones in a |
| | | genotype-independent manner, would not be affected by the presence of |
| | | a cone misgene that maintains the degenerative process. |
| | | RP is a progressive disease involving both eyes. Presently, there is no |
| | | specific treatment and therapeutic approaches are mainly conservative; |
| | | therefore, no active comparators are available. PRP treatment will be |
| | | applied to one eye, and the comparator will be the contralateral eye. |
| | | On the other hand, dry age-related macular degeneration (d-AMD) is a |
| | | multifactorial disease in which genetic and environmental factors are |
| | | involved. More and more genetic alterations have been described in |
| | | literature, although the environmental factors have been proved to play a |
| | | key role in the phenotypic manifestations. |
| Objectives | 7 | The aim of this study is to test the hypothesis that the subretinal injection |
| | | of CB-PRP is safe and exerts a short-term rescue of cone function in RP |
| | | with primary rod degeneration and secondary cone degeneration and d- |
| | | AMD. |
| | | This study will provide the first-in-human proof-of-principle that the |
| | | subretinal implant of a standard jelly CB-PRP is safe and can rescue |
| | | secondary cone photoreceptor function in RP and d-AMD. |
| Trial design | 8 | Prospective interventional NON-randomized single-center trial design |
| | | (framework: exploratory) |

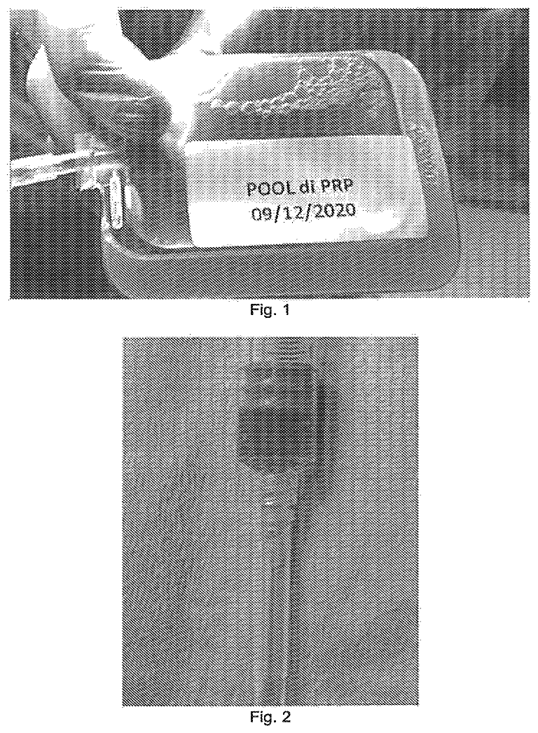

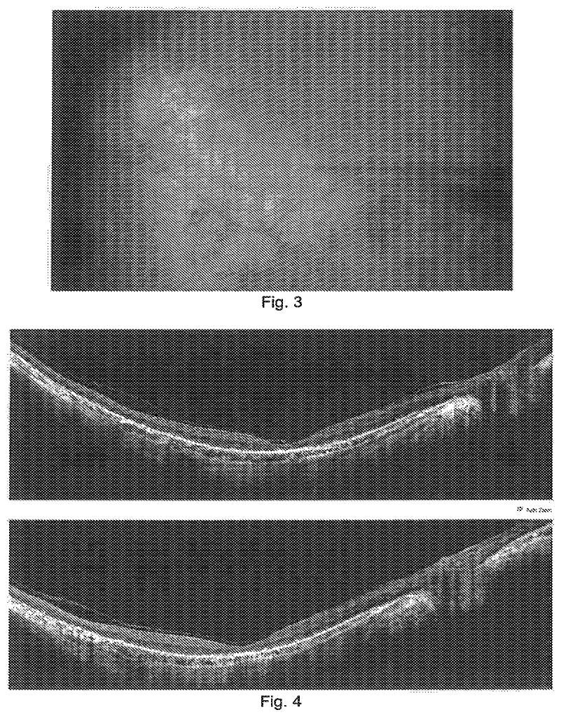

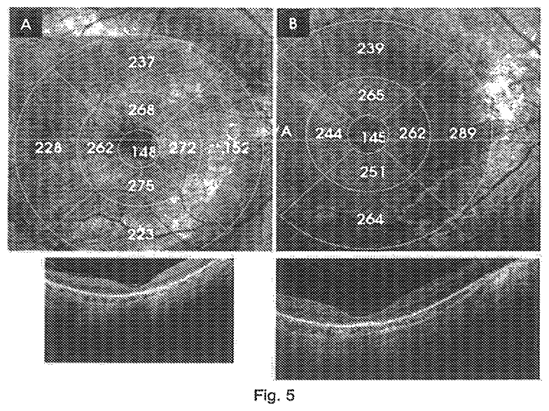

Methods: Participants, interventions, and outcomes

| | | |
|---|---|---|
| Study setting | 9 | The study will be conducted at the Fondazione Policlinico Agostino |
| | | Gemelli, IRCCS, Rome, Italy. |
| Eligibility criteria | 10 | A group of 15 RP patients, selected from 480 RP patients (age range: 15- |
| | | 68 years), representing all genetic types of RP and currently followed at |
| | | our outpatient clinic will be enrolled in the study. These patients have rod- |
| | | specific gene mutations: Rhodopsin PRPF8 and PRPF31mutations |
| | | (responsible of AD RP), and phosphodiesterase (PFE6B) mutation |
| | | (responsible of AR RP). A full general and ophthalmologic examination |
| | | (including detailed family history, anterior segment biomicroscopy, |
| | | corrected ETDRS visual acuity, direct and indirect ophthalmoscopy, |
| | | intraocular pressure measurement) will be performed on each patient. |
| | | Patients will meet all the following inclusion criteria: |
| | | 1. Typical RP with a rod-cone pattern of retinal dysfunction, as |
| | | determined by standard Ganzfeld electroretinography and dark- |
| | | adapted fundus perimetry, and classic fundus appearance, |
| | | 2. Relatively preserved central retinal function (visual field by |
| | | Goldmann V/4e > 30°, corrected ETDRS visual acuity >20/40), |
| | | 3. Known genotype |
| | | 4. At feast four follow-up clinical examination over the past three |
| | | years. |
| | | 5. No or minimal ocular media opacities, |
| | | 6. No concomitant ocular (e.g. glaucoma, amblyopia) or systemic |
| | | diseases. |
| | | Exclusion criteria are one or more of the following: |
| | | 1. Age <18 years |
| | | 2. Pregnant status |
| | | 3. Previous inflammatory/infectious events involving eyes |
| Interventions | 11a | Surgical Procedure: |
| | | The surgical approach will be a 23 Gauge (G) High Speed Pars Plana |
| | | Vitrectomy (10.000 cuts/minute) combined with cataract |
| | | phacoemulsification |
| | | and PC-IOL implantation at the same time when required. Constellation |
| | | Vision System (Alcon Laboratories, Inc., Fort Worth, TX) will be the |
| | | instrument used for all procedures. All patients will receive peribulbar |
| | | anesthesia 30 minutes before surgery (10 ml of ropivacaine combined |
| | | with hyaluronidase 300 Ul). The periocular skin and inferior conjunctival |
| | | fornix will be prepared with 5% povidone-iodine. As previously described, |
| | | before introducing 23G cannulas with trocars, the conjunctiva is partially |
| | | moved to misalign the conjunctiva from the sclera and an angled incision |
| | | weill be performed. Three valved cannulas will be located at 2 and 11 |
| | | o'clock for the service sclerotomies and at 7 o'clock for infusion. They will |
| | | be placed at 3.5 or 4.0 mm for pseudophakia or phakia, respectively. |
| | | Cataract surgery, if required, will be performed through 2 corneal |
| | | incisions: a principal incision (tunnel) of 2.2 mm and a service incision of |
| | | 0.9 mm. The phacoemulsification technique will be used for cataract |
| | | extraction. A singular corneal suture (Ethicon 10-0; Ethicon Inc., San |
| | | Angelo, TX) on the corneal tunnel will be applied if necessary. All eyes |
| | | will receive detachment and removal of the posterior hyaloid membrane if |
| | | it is not separated from the retinal layers below. Core vitrectomy will be |
| | | then performed. Diluted triamcinolone acetonide will be always used to |
| | | dye residual vitreous and peel the epiretinal |
| | | membrane. Through a 41G cannula a sub-retinal injection of 0.5 ml of |
| | | CB-PRP (cord blood- platelet rich plasma) will be done underneath the |
| | | retina. The site of the injection will be chosen according to the surgeons' |
| | | choices and intra-operative conditions. |
| | | Then a complete vitrectomy of the vitreous will be done using Re-Sight |
| | | inverting images and scleral indentation. Peripheral retinal |
| | | photocoagulation will be performed if retinal tears, holes, or |
| | | rhegmatogenous degenerations will be observed. Filtered air will be used |
| | | as endotamponade. After cannula removal, sclerotomies will be checked, |
| | | and scleroconjunctival closures will be applied if needed by bipolar |
| | | diathermy or a single suture (Vicryl 7-0; Ethicon Inc.). After surgery, |
| | | patients will be recommended to maintain a prone position for several |
| | | hours per 3 days. |
| | | Cord blood -Platelet Rich Plasma production. |
| | | CB-PRP is a standardized blood product for non-transfusion use |
| | | produced according to standardized procedures defined by the Italian |
| | | regulation on blood product for transfusion and non-transfusion use |
| | | (Decreto del Ministero della Salute, 2 novembre 2015: Disposizioni |
| | | relative ai requisiti di qualità e sicurezza del sangue e degli |
| | | emocomponenti. GU n. 300 del 28 dicembre 2015; Decreto del Ministero |
| | | della Salute, 19 agosto 2019: Modifiche al decreto 2 novembre 2015, |
| | | recante: «Disposizioni relative ai requisiti di qualita' e sicurezza del |
| | | sangue e degli emocomponenti». GU n. 226 del 26 Sep. 2019 . Source |
| | | material for PRP manufacturing are cord blood units collected at Cord |
| | | Blood Bank (CBB) UNICATT of Fondazione Policlinico Gemelli IRCCS. |
| | | These units are allogeneic solidary donations for hematological patients |
| | | candidate to hematopoietic stem cell transplantation. In this field, the use |
| | | of cord blood represents a therapeutic practice consolidated by decades |
| | | of experience. In order to be transplanted, cord blood units must contain |
| | | an adequate amount of hematopoietic progenitors. Those units not |
| | | fulfilling the established cell thresholds for transplantation are not stored |
| | | and, provided donors consent, can be destined to additional clinical and |
| | | research uses. According to the abovementioned Italian regulation, cord |
| | | blood donors are given the ability to donate after a meticulous medical |
| | | counseling. Moreover, soon after collection, infectious screening tests are |
| | | performed on the mother blood sample (HIV, HBV, HCV and syphilis) or |
| | | on the unit (microbial test). Only units with proved negative tests will be |
| | | utilized for this study. |
| | | CB-PRP will consist of a pool of 15 units. Soon after the collection, each |
| | | unit will be submitted to a soft-spin centrifugation to obtain the PRP. The |
| | | platelet count will be normalized in all PRP at 1 × 10\hat{ }9/L platelets, through |
| | | an hard-spin centrifugation of PRP and subsequent removal of the |
| | | platelet-poor plasma excess. Recovered CB-PRP will be than store at −80° |
| | | C. until microbial tests are proved negative. In order to avoid eventual |
| | | disparities in growth factors concentration among different units, 15 sterile |
| | | CB-PRP will be thawed, pooled, divided in 1 ml aliquots in sterile sealed |
| | | tubes and then stored again at −80° C. until use. This will make intervention |
| | | homogeneous for all patients participating to the study. All necessary |
| | | steps to prepare the CB-PRP will occur in sealed bags, and sterile |
| | | connections will be used to transfer the CB-PRP from a bag to another, or |
| | | to store aliquots of the CB-PRP pool. An additional microbial test will be |
| | | performed on the final product. |
| | 11b | The occurrence of sudden worsening of disease and/or bacterial |
| | | infections and/or inflammatory reactions reasonably connected to the |
| | | CB-PRP treatment in four patients will constitute a stopping rule. |
| | 11c | NA |
| | 11d | All available therapies currently adopted for RP are allowed. |
| Outcomes | 12 | Primary |
| | | Optical Coherence Tomography Structural and Angiography differences |
| | | studied with image subtraction software at at 1, 3, 6 and 12 months |
| | | Secondary |
| | | ETDRS visual acuity at 1, 3, 6 and 12 months |
| | | micro perimetry (Nidek MP1 or MAIA) 6 and 12 months |
| | | Ganzfeld flicker and focal cone electroretinograms (fERGs; Falsini et al, |
| | | 2000) 6 and 12 months |
| | | contrast sensitivity (MARS tables) 6 and 12 months |
| | | Goldmann perimetry. 6 and 12 months |
| | | Explanation of the clinical relevance of chosen efficacy and harm |
| | | outcomes is strongly recommended |
| Participant | 13 | Schedule of enrolment, interventions assessments, and visits for |
| timeline | | participants, is summarized in FIG. 1. |
| | | (in preparation) |
| Sample size | 14 | Details of sample size calculation and statistical analysis. |
| | | In all statistical analyses, SEs and 95% confidence intervals of the means |
| | | will be considered for between-group comparisons. Ganzfeld flicker and |
| | | FERG amplitude data will be log10 transformed to better approximate |
| | | normal distribution. |
| | | Sample size estimates of patients for this study are based on a previous |
| | | investigation (Falsini et al., 1999) where the between- and within-subjects |
| | | variability (expressed as data SD) of visual acuity, microperimetry, |
| | | Ganzfeld flicker and fERG parameters was determined in RP patients. |
| | | For the ERG analysis, assuming between- and within-subjects SDs in |
| | | FERG amplitude and phase of 0.2 logμV and 30 degrees, respectively, |
| | | the sample sizes of patients' eyes assigned to both treatment and control |
| | | groups will provide a power of 80%, at an α = 0.05, for detecting in each |
| | | group a test-retest difference (i.e. 180 days minus baseline test) of 0.1 |
| | | logμV (SD: 0.1) and 30 degrees (SD: 20) in amplitude and phase, |
| | | respectively. Given the absolute mean amplitude and phase values of the |
| | | patients' fERG, these differences are considered as clinically meaningful, |
| | | since they correspond approximately to a 25-30% change in either |
| | | amplitude or phase. The patients' sample size also provides a power of |
| | | 90%, at an α = 0.05, for detecting between-group differences in FERG |
| | | amplitude and phase changes (i.e. 180 days minus baseline test) of 0.15 |
| | | logμV (SD: 0.1) and 40 degrees (SD: 20), respectively. |
| | | In the main analyses, a p < 0.05 will be considered statistically significant |
| | | for the multivariate and repeated measures ANOVA. A more conservative |
| | | p value of 0.02 will be adopted for multiple comparisons. |
| Recruitment | 15 | Strategies for achieving adequate participant enrolment to reach target |
| | | sample size |

Methods: Assignment of interventions (for controlled trials)

| | | |
|---|---|---|
| Allocation: | | |
| Sequence | 16a | Not applicable |
| generation | | |
| Allocation | 16b | Not applicable |
| concealment | | |
| mechanism | | |
| Implementation | 16c | Not applicable |
| Blinding | 17a | Tests will be performed by operators that will be masked as to the treated |
| (masking) | | eye of each patient. Tests will be conducted in both eyes. The fellow, |
| | | untreated eye of each patient will serve as control |
| | 17b | Not applicable |

Methods: Data collection, management, and analysis

| | | |
|---|---|---|
| Data collection | 18a | The following data will be collected at enrolment: |
| methods | | General ophthalmological examination |
| | | Optical coherence tomography |
| | | Visual field |
| | | electroretinography |
| | | The following data will be collected at treatment: |
| | | General ophthalmological examination |
| | | Optical coherence tomography |
| | | Visual field |
| | | electroretinography |
| | | Follow-up visits are scheduled at days 0 (baseline) 7, 15, 30, 60, 90, |
| | | 180 . . . |
| | | AI each visit the following examinations will be carried out: |
| | | General ophthalmological examination |
| | | Optical coherence tomography |
| | | Visual field |
| | | electroretinography |
| | | Plans for assessment and collection of outcome, baseline, and other trial |
| | | data, including any related processes to promote data quality (eg, |
| | | duplicate measurements, training of assessors) and a description of study |
| | | instruments (eg, questionnaires, laboratory tests) along with their |
| | | reliability and validity, if known. Reference to where data collection forms |
| | | can be found, if not in the protocol |
| | 18b | Plans to promote participant retention and complete follow-up, including |
| | | list of any outcome data to be collected for participants who discontinue |
| | | or deviate from intervention protocols |
| Data | 19 | Data will be collected by personal directly involved in the study, delegated |
| management | | by the Principal Investigators. Patient data will be deidentified and made |
| | | anonymous through an association with specified codes |
| Statistical | 20a | Statistical methods for analysing primary and secondary outcomes. |
| methods | | Reference to where other details of the statistical analysis plan can be |
| | | found, if not in the protocol |
| | | See Statistical Plan |
| | 20b | Methods for any additional analyses (eg, subgroup and adjusted |
| | | analyses) |
| | 20c | Definition of analysis population relating to protocol non-adherence (eg, |
| | | as randomised analysis), and any statistical methods to handle missing |
| | | data (eg, multiple imputation) |

Methods: Monitoring

| | | |
|---|---|---|
| Data monitoring | 21a | Composition of data monitoring committee (DMC); summary of its role |
| | | and reporting structure; statement of whether it is independent from the |
| | | sponsor and competing interests; and reference to where further details |
| | | about its charter can be found, if not in the protocol. Alternatively, an |
| | | explanation of why a DMC is not needed |
| | 21b | Description of any interim analyses and stopping guidelines, including |
| | | who will have access to these interim results and make the final decision |
| | | to terminate the trial |
| Harms | 22 | Plans for collecting, assessing, reporting, and managing solicited and |
| | | spontaneously reported adverse events and other unintended effects of |
| | | trial interventions or trial conduct |
| Auditing | 23 | Frequency and procedures for auditing trial conduct, if any, and whether |
| | | the process will be independent from investigators and the sponsor |

Ethics and dissemination

| | | |
|---|---|---|
| Research ethics | 24 | It is the responsibility of the investigator to have prospective approval of |
| approval | | the study protocol, protocol amendments, informed consent forms, and |
| | | other relevant documents, eg, advertisements, if applicable, from the |
| | | IRB/IEC. All correspondence with the IRB/IEC should be retained in the |
| | | Investigator File. The principal investigator agrees to provide the Ethics |
| | | Committee/IRB with all appropriate material, including a copy of the |
| | | informed consent. The study will not be initiated until the investigator |
| | | obtains written approval of the research plan and the informed consent |
| | | document from the appropriate Ethics Committee/IRB. |
| Protocol | 25 | Any protocol modifications will be communicated to the Ethics Committee. |
| amendments | | |
| Consent or | 26a | It is the responsibility of the Principal Investigator or his delegates to obtain |
| assent | | a written informed consent from each subject. The Principal Investigator or |
| | | his delegates will explain the nature of the trial, its purpose, the procedures |
| | | involved, the expected duration, the potential risks and benefits involved |
| | | and any discomfort it may entail and provide the subject with a copy of the |
| | | information sheet. The subject will be given sufficient time to consider the |
| | | trial before deciding whether to participate. Each subject must be informed |
| | | that participation in the trial is voluntary and that he/she may withdraw from |
| | | the trial at any time and that withdrawal of consent will not affect his/her |
| | | subsequent medical treatment or relationship with the treating physician. |
| | | All patients are to provide written informed consent in accordance with |
| | | applicable laws of the country. The patient will sign and date the informed |
| | | consent form before he/she enters the study (i.e. before any study related |
| | | activity). |
| | 26b | Additional consent provisions for collection and use of participant data |
| | | and biological specimens in ancillary studies, if applicable |
| | | NA |
| Confidentiality | 27 | The patient's confidentiality will be maintained and will not be made publicly |
| | | available to the extent permitted by the applicable laws and regulations. A |
| | | sequential identification number will be automatically attributed to each |
| | | patient enrolled in the study. This number will identify the patient and must |
| | | be included on all case report forms. |
| Declaration of | 28 | The study investigators have not competing interests. |
| interests | | |
| Access to data | 29 | The PI or his delegates will have access to trial dataset. Data will be filed |
| | | in a PC accessible only to investigators through personal passwords. |
| | | Those individuals who can access documentation will take every |
| | | precaution to keep the identity of the subjects as reserved information, in |
| | | accordance with relevant applicable legislation. |
| Ancillary and | 30 | Provisions, if any, for ancillary and post-trial care, and for compensation |
| post-trial care | | to those who suffer harm from trial participation. |
| | | The CB-PRP is a blood product whose utilization is regulated by the Italian |
| | | transfusion regulation. The study is promoted by Fondazione Policlinico |
| | | Gemelli IRCCS (no-profit), which represents clinicians that, in routine |
| | | practice, bear responsibility for the care of patients, according to the |
| | | National Health System. Clinicians will follow the features expected by the |
| | | Ministerial Decree of Dec. 17, 2000, published on GU no. 43 of Feb.22, 2005 |
| | | which regulates non-commercial clinical trials, aiming the improvement of |
| | | clinical practice. Patients are treated according to the best standards within |
| | | their rights as citizens under the National Health System. Therefore, there |
| | | is no need for any additional specific procedures of insurance policy, other |
| | | than the already existing ones provided by the National Health System. |
| Dissemination | 31a | Investigators will communicate trial results to participants, healthcare |
| policy | | professionals, the public, and other relevant groups, reporting The |
| | | Principal Investigator or his delegates allows the Ethics Committee to |
| | | have free access to and to conduct the relevant verification of all the |
| | | original documentation of the study, including the informed consent forms |
| | | signed by the subjects enrolled into the study, the relevant patient files |
| | | and/or out-patient files. Their findings in scientific publications |
| | 31b | Authorship will be established according to the International committee of |
| | | medical journal editors |
| | 31c | Public access to the full protocol, participant-level dataset, and statistical |
| | | codes will be granted to the Ethics Committee, or, after Ethics Committee |
| | | authorization, to the members of the scientific community who will be |
| | | interest in detailed analysis of trial results. |
| Appendices | | |
| Informed consent | 32 | Participants will be given an informed consent form specific for this study. |
| materials | | |
| Biological | 33 | Plans for collection, laboratory evaluation, and storage of biological |
| specimens | | specimens for genetic or molecular analysis in the current trial and for |
| | | future use in ancillary studies, if applicable |
| | | NA |

### REFERENCES

1. Galli-Resta L, Falsini B, Rossi G, et al. Bilateral Symmetry of Visual Function Loss in Cone-Rod Dystrophies. Invest Ophthalmol Vis Sci. 2016; 57(8):3759-3768.
2. Rosenblatt T R, Vail D, Saroj N, Boucher N, Moshfeghi D M, Moshfeghi A A. Increasing Incidence and Prevalence of Common Retinal Diseases in Retina Practices Across the United States. Ophthalmic Surg Lasers Imaging Retina. 2021 Jan. 1; 52(1):29-36.
3. Narayan D S, Wood J P, Chidlow G, Casson R J. A review of the mechanisms of cone degeneration in retinitis pigmentosa. Acta Ophthalmol. 2016; 94(8):748-754.
4. Cinici E, Caglar O, Arslan M E, Dilekmen N, Utlu B, Mardinoglu A, Turkez H. Targeted Gene Candidates for Treatment and Early Diagnosis of Age-Related Macular Degeneration. Biomed Res Int. 2021 Feb. 2; 2021:6620900.
5. Saari J C. Vitamin A and Vision. Subcell Biochem. 2016; 81:231-259.
6. Li S, Sato K, Gordon W C, Sendtner M, Bazan N G, Jin M. Ciliary neurotrophic factor (CNTF) protects retinal cone and rod photoreceptors by suppressing excessive formation of the visual pigments. J Biol Chem. 2018 Sep. 28; 293(39):15256-15268. doi: 10.1074/jbc.RA118.004008. Epub 2018 Aug. 16. PMID: 30115683; PMCID: PMC6166737.
7. Wen R, Tao W, Li Y, Sieving P A. CNTF and retina. Prog Retin Eye Res. 2012; 31(2):136-151.
8. Guadagni V, Novelli E, Piano I, Gargini C, Strettoi E. Pharmacological approaches to retinitis pigmentosa: A laboratory perspective. Prog Retin Eye Res. 2015; 48:62-81.
9. Sieving P A, Caruso R C, Tao W, et al. Ciliary neurotrophic factor (CNTF) for human retinal degeneration: phase I trial of CNTF delivered by encapsulated cell intraocular implants. Proc Natl Acad Sci USA. 2006; 103(10):3896-3901. doi:10.1073/pnas.0600236103;
10. Pilli S, Zawadzki R J, Telander D G. The dose-dependent macular thickness changes assessed by fd-oct in patients with retinitis pigmentosa treated with ciliary neurotrophic factor. Retina. 2014; 34(7):1384-90.
11. Falsini B, Iarossi G, Chiaretti A, et al. NGF eye-drops topical administration in patients with retinitis pigmentosa, a pilot study. J Transl Med. 2016; 14:8. Published 2016 Jan. 9. doi:10.1186/s12967-015-0750-3;
12. Kim J B, Lad E M. Therapeutic Options Under Development for Nonneovascular Age-Related Macular Degeneration and Geographic Atrophy. Drugs Aging. 2021 January; 38(1):17-27.
13. https://clinicaltrials.gov/ct2/show/NCT03919019?cond=dry+amd&draw=2&rank=3
14. https://clinicaltrials.gov/ct2/show/NCT03305029?cond=dry+amd&draw=2&rank=7
15. Mandai M, Watanabe A, Kurimoto Y, Hirami Y, Morinaga C, Daimon T, Fujihara M, Akimaru H, Sakai N, Shibata Y, Terada M, Nomiya Y, Tanishima S, Nakamura M, Kamao H, Sugita S, Onishi A, Ito T, Fujita K, Kawamata S, Go M J, Shinohara C, Hata K I, Sawada M, Yamamoto M, Ohta S, Ohara Y, Yoshida K, Kuwahara J, Kitano Y, Amano N, Umekage M, Kitaoka F, Tanaka A, Okada C, Takasu N, Ogawa S, Yamanaka S, Takahashi M. Autologous Induced Stem-Cell-Derived Retinal Cells for Macular Degeneration. N Engl J Med. 2017 Mar. 16; 376(11):1038-1046. doi: 10.1056/NEJMoa1608368. PMID: 28296613.
16. Alio J L, Rodriguez A E, WróbelDudzińska D. Eye platelet-rich plasma in the treatment of ocular surface disorders. Curr Opin Ophthalmol. 2015; 26(4):325-32;
17. Versura P, Profazio V, Buzzi M, Stancari A, Arpinati M, Malavolta N, Campos E C. Efficacy of standardized and quality-controlled cord blood serum eye drop therapy in the healing of severe corneal epithelial damage in dry eye. Cornea. 2013 April; 32(4):412-8;
18. Platelet-rich plasma injections for wound healing and tissue rejuvenation: a review of clinical effectiveness, cost-effectiveness and guidelines. Ottawa: CADTH; 2017 June (CADTH rapid response report: summary with critical appraisal). Available at https://www.cadth.ca/sites/default/files/pdf/htis/2017/RC0885%20PRP %20Injections %20Final.pdf. (Last accessed Feb. 5, 2020);
19. Valentini C G, Nuzzolo E R, Bianchi M, Orlando N, Iachininoto M G, Pinci P, Teofili L. Cord Blood Platelet Lysate: In Vitro Evaluation to Support the Use in Regenerative Medicine. Mediterr J Hematol Infect Dis. 2019 Jan. 1; 11(1).
20. Falsini B., Bush R A, Sieving P A: Neuroprotection. In Ryan's Retina, 6th edition, Schachat A P, Wilkinson C P, Hinton D R, Sadda S R and Wiedemann P eds, Ch 39, 2017.
21. Di Marco S, Riccitelli S, Di Paolo M, et al. Cord Blood Serum (CBS)-Based Eye Drops Modulate Light-Induced Neurodegeneration in Albino Rat Retinas. Biomolecules. 2020; 10(5):E678. Published 2020 Apr. 28.
22. Abed E, Placidi G, Calandriello L, et al. Correlation of Macular Focal Electroretinogram with Ellipsoid Zone Extension in Stargardt Disease. J Ophthalmol. 2017; 2017:3643495.
23. Falsini B, Galli-Resta L, Fadda A, et al. Long-term decline of central cone function in retinitis pigmentosa evaluated by focal electroretinogram. Invest Ophthalmol Vis Sci. 2012; 53(12):7701-7709. Published 2012 Nov. 19.
24. Minnella A M, Pagliei V, Savastano M C, et al. Swept source optical coherence tomography and optical coherence tomography angiography in pediatric enhanced S-cone syndrome: a case report. J Med Case Rep. 2018; 12(1):287. Published 2018 Oct. 3.
25. Savastano M C, Ziccardi L, Zinzanella G, Falsini B, Minnella A M. Sequential spectral domain optical coherence tomography imaging of an eye after successful removal of subfoveal perfluorcarbon liquid collection. Retin Cases Brief Rep. 2014; 8(3):215-218.
26. Savastano M C, Minnella A M, Tamburrino A, Giovinco G, Ventre S, Falsini B. Differential vulnerability of retinal layers to early age-related macular degeneration: evidence by SD-OCT segmentation analysis. Invest Ophthalmol Vis Sci. 2014; 55(1):560-566. Published 2014 Jan. 29.
27. Savastano A, Lenzetti C, Finocchio L, Bacherini D, Giansanti F, Tartaro R, Piccirillo V, Savastano M C, Virgili G, Rizzo S. Combining cataract surgery with 25-gauge high-speed pars plana vitrectomy: A prospective study. Eur J Ophthalmol. 2021 March; 31(2):673-678. doi: 10.1177/1120672120902030. Epub 2020 Jan. 23. PMID: 31973558.
28. Savastano A, Savastano M C, Barca F, Petrarchini F, Mariotti C, Rizzo S. Combining cataract surgery with 25-gauge high-speed pars plana vitrectomy: results from a retrospective study. Ophthalmology. 2014 January; 121(1):299-304.
29. Johnson M H, Cohen J. Reprogramming rewarded: the 2012 Nobel Prize for Physiology or Medicine awarded to John Gurdon and Shinya Yamanaka. Reprod Biomed Online. 2012 December; 25(6):549-50.
30. Mandai M, Watanabe A, Kurimoto Y, Hirami Y, Morinaga C, Daimon T, Fujihara M, Akimaru H, Sakai N, Shibata Y, Terada M, Nomiya Y, Tanishima S, Nakamura M, Kamao H, Sugita S, Onishi A, Ito T, Fujita K, Kawamata S, Go M J, Shinohara C, Hata K I, Sawada M, Yamamoto M, Ohta S, Ohara Y, Yoshida K, Kuwahara J, Kitano Y, Amano N, Umekage M, Kitaoka F, Tanaka A, Okada C, Takasu N, Ogawa S, Yamanaka S, Takahashi M. Autologous Induced Stem-Cell-Derived Retinal Cells for Macular Degeneration. N Engl J Med. 2017 Mar. 16; 376(11):1038-1046.
31. https://clinicaltrials.gov/ct2/show/NCT04636853?term=savastano+alfonso&draw=2&r ank=1.
32. Anitua E, Muruzabal F, Tayebba A, Riestra A, Perez V L, Merayo-Lloves J, Orive G. Autologous serum and plasma rich in growth factors in ophthalmology: preclinical and clinical studies. Acta Ophthalmol. 2015 December; 93(8):e605-14.

1. A composition comprising cord blood platelet-rich plasma (CB-PRP).

2. The composition according to claim 1, wherein said composition is characterized by a platelet concentration of 1×10⁶ platelets/μL.

3. The composition according to claim 1, further comprising one or more platelet derived growth factor comprising Granulocyte colony-stimulating factor (G-CSF), Granulocyte-Macrophage Colony-Stimulating Factor (GM-CSF), Vascular-Endothelial Growth Factor (VEGF), Fibroblast Growth Factors (FGF), and/or Platelet Derived Growth Factor (PDGF).

4. The composition according to claim 1, further comprising at least one pharmaceutically acceptable excipient and/or carrier.

5. The composition according to claim 1, in the form of a solution, suspension, gel, eye drops.

6.-9. (canceled)

10. A method for the treatment of an ocular disease in a subject in need thereof, which method comprises:
administering to said subject an effective amount of a composition according to claim 1.

11. The method according to claim 10, wherein said ocular disease is an ocular surface disease (OSD) or a posterior segment eye disease (PSED).

12. The method according to claim 10, wherein said ocular disease is selected from a group comprising retinitis pigmentosa (RP), dry age-related macular degeneration (d-AMD), glaucoma disease and corneal neurotrophic ulcers.

13. The method according to any claim 10, wherein said administering is carried out by way of a topic and/or intraocular administration.

14. The method according to claim 10, wherein said composition is administered at a dose regimen of 1 drop of said composition for 6 times daily for 7 days.

15. The method according to claim 10, wherein said administration is carried out by way of sub-retinal injection and/or intravitreal injection.

16. The method according to claim 10, wherein said composition is administered:
by way of intravitreal injection at a dose regimen of 1 injection of 0.1 mL of said composition monthly for 6 months, wherein said injection is optionally repeated; or
by way of sub-retinal injection at a dose regimen of 1 injection of 0.5 mL of said composition, wherein said sub-retinal injection is optionally followed by intravitreal injection.

17. The method according to any one of claim 16, wherein said subject undergoes cataract surgery and/or vitrectomy prior and/or after said administration.

18. The method according to claim 16, wherein said subject undergoes least one of the following peripheral retinal photocoagulation; scleroconjunctival closures by means of bipolar diathermy or a single suture, after said administration

19. The method according to claim 16, wherein said subject is subjected to post-administration monitoring at one or more period of time, by at least one of the following techniques: optical coherence tomography, visual field, electroretinography.

20.-22. (canceled)
